(19) United States
(12) Patent Application Publication
(10) Pub. No.: US 2016/0244727 A1
(43) Pub. Date: Aug. 25, 2016

(54) ARTIFICIAL MICROVASCULAR DEVICE AND METHODS FOR MANUFACTURING AND USING THE SAME

(71) Applicant: The Charles Stark Draper Laboratory, Inc., Cambridge, MA (US)

(72) Inventors: Jeffrey T. Borenstein, Newton, MA (US); James Ching-Ming Hsiao, Somerville, MA (US); Mark E. Keegan, Littleton, MA (US); Ernest S. Kim, Cambridge, MA (US)

(21) Appl. No.: 15/145,286

(22) Filed: May 3, 2016

Related U.S. Application Data
(63) Continuation of application No. 12/398,311, filed on Mar. 5, 2009.
(60) Provisional application No. 61/037,196, filed on Mar. 17, 2008.

(51) Int. Cl. C12N 5/071; C12M 1/00; C12M 1/34; C12M 1/12; C12M 3/06

(52) U.S. Cl. CPC C12N 5/0691; C12M 25/06; C12M 23/16; C12M 41/40; C12M 25/02; C12M 23/40; C12N 2527/00

(57) ABSTRACT

Artificial microvascular devices may include a polymer scaffold that defines a channel with a distensible wall.

### CROSS-REFERENCE TO RELATED APPLICATION

This application is a continuation of, and claims priority to, U.S. patent application Ser. No. 12/398,311, filed on Mar. 5, 2009, which claims priority to and the benefit of U.S. Provisional Patent Application No. 61/037,196, filed on Mar. 17, 2008, both of which are incorporated herein by reference in their entirety.

### FIELD OF THE INVENTION

In various embodiments, the invention relates to microdevices for biomedical applications, and, more particularly, to an artificial microvascular network for use as a tool in drug discovery and drug safety testing.

### BACKGROUND

Drug discovery and drug safety testing are conventionally performed through cell culture and tissue culture assays, tests with animal models, and human clinical trials. These methods, however, typically suffer from several disadvantages. In particular, in using these methods, it is often difficult to: i) provide the statistical significance necessary to firmly establish the efficacy or safety of a drug, ii) directly correlate safety and efficacy results with specific mechanisms, and/or iii) encompass the range of genetic variations expected in the patient population for a drug. In addition, high cost is often associated with animal and human clinical trials, and human clinical trial participants may be endangered.

Microfluidic devices provide means for studying the interaction of cells and cell cultures with biological and chemical species in vitro, and, consequently, without risk to human patients. Since they are usually scalable to high-throughput equipment, they enable systematic tests and the obtainment of statistically meaningful data. Microfluidic devices typically comprise a network of channels embedded in a polymer scaffold material. To model biological tissues, cells can be plated within the channels. For example, a network of microchannels with diameters ranging from microns to millimeters can be lined with endothelial and smooth muscle cells to provide an artificial vascular network of realistic dimensions. However, customary artificial vasculoid structures differ from their physiological counterparts in important aspects. For example, artificial channels usually have rigid, impermeable walls, whereas biological vessels, such as blood or lymphatic vessels, can respond dynamically to mechanical stresses by stretching and bending, and allow for the transfer of macromolecules through the vascular walls. Moreover, artificial devices greatly simplify vascular structure by severely limiting the number of cell types used, sometimes to only one type, and by failing to establish structures or conditions that induce cell organization similar to that found in real vasculoids.

Accordingly, there is a need for artificial vascular networks that resemble physiological vascular networks more closely, and consequently enable drug efficacy and safety testing with cells in a more realistic microenvironment.

### SUMMARY OF THE INVENTION

Embodiments of the present invention provide structures and manufacturing methods for artificial microvascular devices that mimic key features of physiological vascular networks. Specifically, various embodiments provide artificial vasculoids with distensible walls and with structural elements facilitating spatially organized cell co-cultures. In various embodiments, the artificial microvascular device is an engineered microfluidic structure that includes at least a scaffold material defining one or more channels therein, which can be populated with animal and/or human vascular cells of various types. The device can be used singly, or combined with other such devices in a multiplexed array. In the following, devices embodying the invention are interchangeably termed microvascular devices, vascular devices, microvascular or vascular networks, or vasculoids, or similarly denominated.

Principal applications of the artificial microvascular device include investigating the efficacy of various chemical or biological compounds against diseases of the cardiovascular system, and studying and identifying compounds associated with adverse effects upon healthy physiologic tissue. For example, commercial applications include using the artificial vasculoid microdevices as tools for early-stage drug discovery and for safety testing of compounds that are in development, already in clinical trials, or approved for use by the United States Food and Drug Administration. Since testing in artificial microvascular devices may utilize human cells, it can project the outcomes of drug use by humans after market introduction more accurately than most animal toxicity and efficacy testing. Also, the artificial vasculoid microdevices described herein may be readily scaled for high-throughput screening, unlike many existing animal or human tests.

In one aspect, embodiments of the invention feature an artificial microvascular device that includes a polymer scaffold defining at least one channel with one or more distensible walls. The scaffold may be transparent, and may be made of or contain one or more of the following materials: polystyrene, polyesteramide, polyglycerol sebacate, polydimethylsiloxane (PDMS), polycarbonate, silk fibroin, polyurethane, polyoctanediol citrate, polydiol citrate, and polycaprolactone. The distensible wall(s) may be made of an elastomeric polymer, such as polyesteramide, polyglycerol sebacate, polydimethylsiloxane, silk fibroin, polyurethane, polyoctanediol citrate, polydiol citrate, and polycaprolactone. In some embodiments, distensibility of the channel wall(s) is achieved by keeping the product of the wall thickness and the elastic modulus of the wall material below about 100 Pascal-meters, preferably below about 50 Pascal-meters. In one embodiment, the wall is made of PDMS and has a thickness of less than about 40 micrometers. The distensible wall may form an outer boundary of the device, or separate the channel from a second, substantially parallel channel or channel segment. In some embodiments, the second channel is in pressure balance with the environment of the device. In alternative embodiments, the second channel is connected to and in fluidic communication with a pressurizing device. In this case, a difference in pressures between the two channels may cause wall distention. The wall between two channels may be fenestrated. In various embodiments, cells are seeded within one or more channels of the microvascular device. The cells may induce distension of the wall from within the channel(s).

In a second aspect, embodiments of the invention feature an artificial microvascular device containing two channels within a polymer scaffold. The two channels are, at least in parts, axially parallel, and are separated by a fenestrated wall. In some embodiments, two types of cells are seeded in the two channels. For example, the first channel may be populated with endothelial cells, and the second channel with other vascular cells, such as, e.g., smooth muscle cells, pericytes, or fibroblasts. Alternatively or additionally, the second channel may be seeded with one or more types of tissue or organ cells, including, but not limited to, neurons, adipocytes, dermal cells, epithelial cells, skeletal muscle cells, bone cells, and hepatocytes. The cells of one type may chemically communicate with the cells of the other type through the fenestrated wall. In certain embodiments, the device may comprise one or more additional channels, separated from the first channel by a distensible wall.

In a third aspect, embodiments of the invention provide a method of manufacturing an artificial microvascular device. The method involves applying a moldable polymer to a master mold having one or more inverse channels, and curing and removing the polymer to create a stamp having an open channel. Further, the method includes covering the channel with a polymer sheet sufficiently thin to render the channel wall that it forms distensible. In some embodiments, the master mold contains two inverse channels, which are in large portions parallel, and which are separated by a wall of less than about 40 microns in thickness. The method may further involve the creation of a second stamp containing an open channel. This second stamp may be affixed to the device upside down, in a manner that aligns the channels in the two stamps and such that the channels in each stamp are separated only by the thin polymer layer therebetween. In general, embodiments of the invention include devices built from an arbitrary number of polymer stamps. In accordance with some embodiments, manufacturing an artificial microvascular device includes connecting tubing into inlet and outlet holes of the device and/or seeding cells in the channel(s).

A fourth aspect of the invention relates to testing the response of artificial microvascular devices, such as those described above, to mechanical stimuli. The method involves the provision of an artificial microvascular device having a channel with a distensible wall and cells seeded therein, and the observation of a response of the distensible wall to a mechanical stimulus. For the purpose of observation, the device may be integrated with an optical apparatus. The mechanical stimulus may be provided by the cells, or it may be applied as one step of the method, e.g., through pressurizing a channel that is separated from the channel under consideration by a distensible wall.

### BRIEF DESCRIPTION OF THE DRAWINGS

The foregoing and other objects, aspects, features, and advantages of the invention will become more apparent and may be better understood by referring to the following description taken in conjunction with the accompanying drawings, in which:

FIG. 1 is a schematic perspective view of a micro-device with one channel according to one embodiment of the invention;

FIG. 2 is a schematic perspective view of a micro-device with a bifurcated channel according to one embodiment of the invention;

FIGS. 3A and 3B are flow diagrams illustrating a method for fabricating microvascular devices according to one embodiment of the invention;

FIGS. 4A-4D are perspective, section, and top views of a microvascular device containing a channel with distensible top and side walls in passive configuration in accordance with one embodiment of the invention;

FIG. 5 is a perspective drawing of an exemplary master mold for the device depicted in FIGS. 4A-4D;

FIGS. 6A-6C are perspective drawings illustrating an exemplary assembly of the device depicted in FIGS. 4A-4D;

FIGS. 7A-7C are perspective and side views of a microvascular device containing a channel with distensible top and side walls in active configuration in accordance with one embodiment of the invention;

FIGS. 8A-8D are perspective drawings illustrating an exemplary assembly of the device depicted in FIGS. 7A-7C;

FIGS. 9A-9D are perspective drawings of a microvascular device containing a channel with four distensible walls in accordance with one embodiment of the invention;

FIGS. 10A and 10B are perspective and side views of a microvascular device with co-culture channels and fenestrated walls in accordance with one embodiment of the invention; and

FIGS. 11A and 11B are perspective and side views of a microvascular device with co-culture channels and fenestrated and distensible walls in accordance with one embodiment of the invention.

### DESCRIPTION

### 1. Basic Device Structure and Fabrication Methods

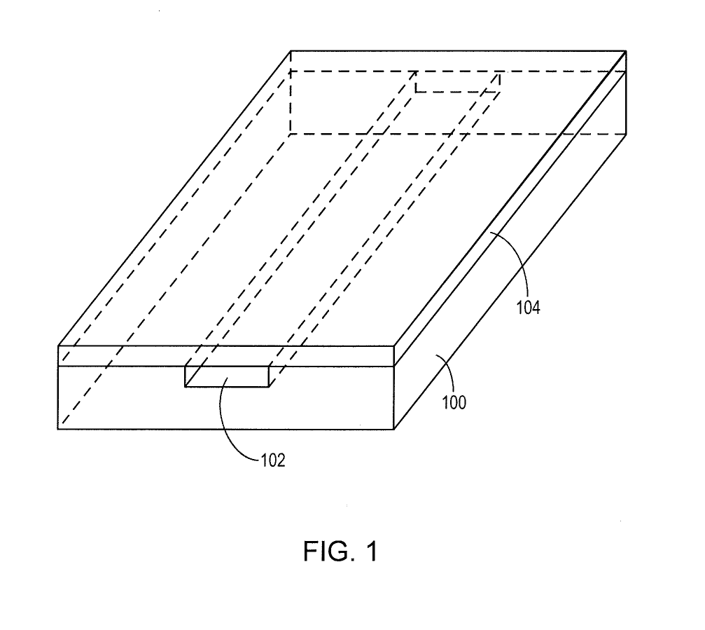

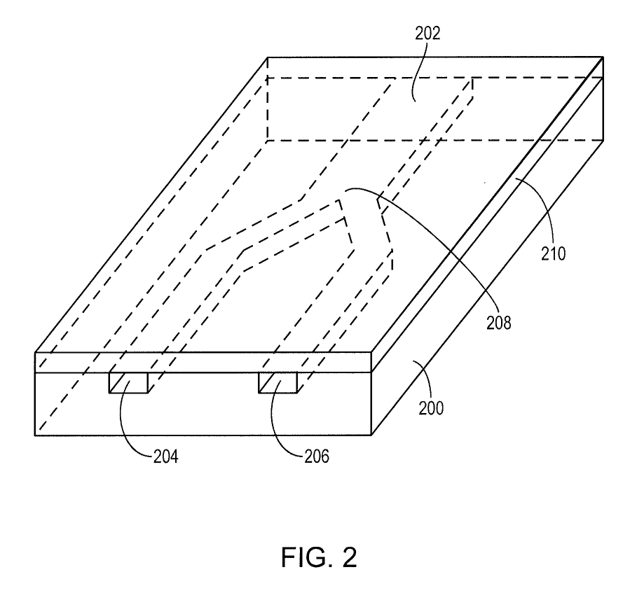

Described herein are exemplary artificial vascular micro-devices for use as a tool in drug discovery and drug safety testing. Such devices can be fabricated from moldable polymers, such as polydimethylsiloxane (PDMS), using, for example, soft lithography. FIG. 1 illustrates a section of an exemplary device made from a layer 100 that defines an open channel 102 with three walls, and a layer 104 that is coupled to layer 100, and which thereby provides a fourth wall to the channel 102. FIG. 2 depicts an exemplary device having a “network” of channels formed in a layer 200. More specifically, a channel 202, which splits into two channels 204 and 206 at a bifurcation 208, is formed in the layer 200. Again, the channels 202, 204, 206 are closed by a top layer 210. Note that, while the foregoing and following illustrations of micro-devices show channels with rectangular cross-sections, this rectangular shape is not an essential characteristic, and devices featuring channels with round and otherwise shaped cross-sections should be considered as falling within the scope of the invention.

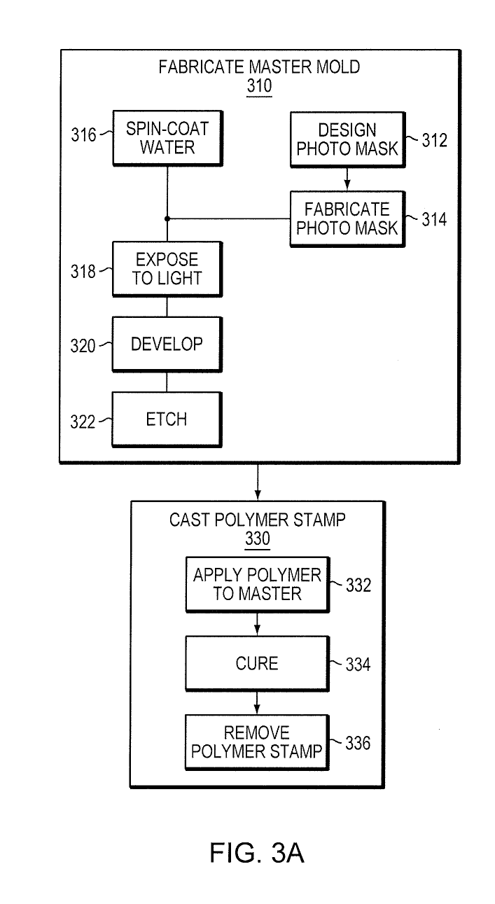

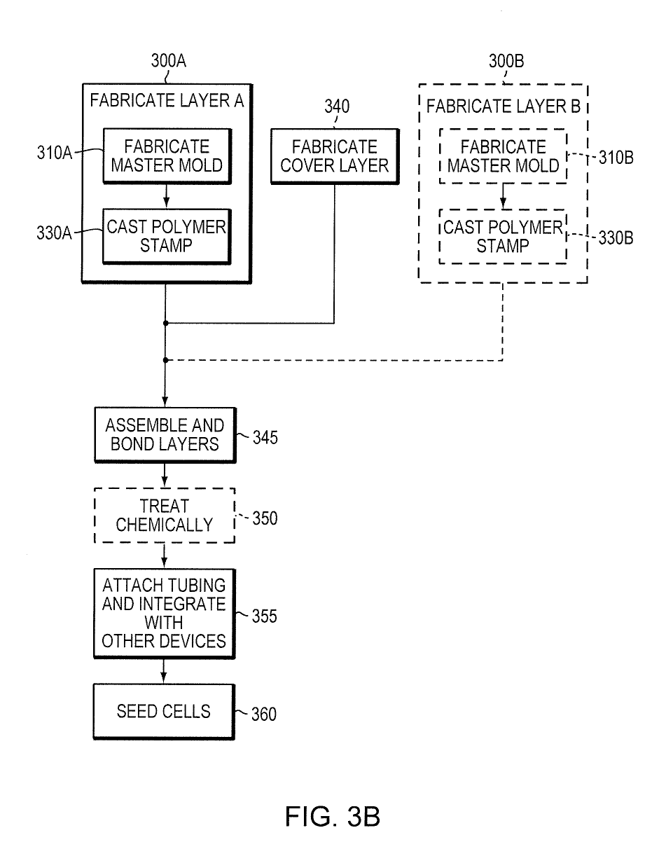

FIGS. 3A and 3B detail an exemplary process sequence that may be followed to manufacture devices embodying the invention, including structures like those shown in FIGS. 1 and 2. Refer first to FIG. 3A, which illustrates a process 300 directed to fabricating a device component such as layer 100 or 200 by means of photolithography and replica molding. In a first sub-processes 310, a master mold featuring a negative relief of the desired structure is fabricated. Sub-process 310 involves, firstly, the design (step 312) and fabrication (step 314) of a photomask that defines the ridges of the master mold, corresponding to the indentations of the final layer, as transparent regions in an otherwise opaque sheet. The mask layout may be defined in a computer drawing, and may then be converted, e.g., with a software package such as Tanner L-Edit, into a Computer-Aided Design (CAD) layout, which is suitable for subsequent physical writing of the mask by electron-beam lithography or a similar technique. In step 316, a substrate wafer, e.g., made from silicon, is spin-coated with a viscous solution of a suitable photoresist, such as, for example, SU-8. Typically, the wafer is spun rapidly, at 1200 to 4800 revolutions per minute, for a time duration ranging from several tens of seconds up to minutes, to produce a uniformly thick layer of photoresist with a thickness of up to tens or even hundreds of micrometers. In step 318, the photomask is placed on the wafer, and the photoresist in the transparent regions of the mask is chemically stabilized by exposure to UV light. Photoresist in non-exposed regions is subsequently removed by exposure to a chemical developing agent (step 320), and the remaining photoresist is hardened at elevated temperatures to form a durable negative relief. In an etching step 322, a chemical agent removes the upmost layer of the substrate in regions that are not protected by photoresist, generating a channel pattern in negative relief in the wafer, which now constitutes the master mold. The photoresist, no longer needed, is afterwards removed from the substrate. In a second sub-process 330, a liquid polymer is casted into the master mold (step 332), cured (step 334), and peeled off (step 336), resulting in a replica mold of the channel-containing layer of the device (e.g., layer 100 or 200). Suitable polymers include, for example, polydimethylsiloxane (PDMS), polystyrene, polyesteramides (PEA), and polyglycerol sebacate (PGS).

Refer now to FIG. 3B, which illustrates how several layers (e.g., layers 100) produced by process 300 are integrated into a functional microvascular device. To produce a simple two-layer device, the channel-comprising layer 100, fabricated in a process 300, may be covered by a thin polymer layer 104, which itself can, for instance, be fabricated by being coated onto a wafer, cured, and peeled off (process 340). For devices containing more than one patterned layer 100, as are described in detail below, process 300 can be carried out for (generally) different layouts; FIG. 3B only indicates two representative processes 300A and 300B. The various layers (e.g., first layer 100, layer 104, and second layer 100) are then assembled and plasma-bonded or otherwise temporarily or permanently attached to each other (step 345). To prepare the device for biological applications, the chemical characteristics of the channel 102 walls may optionally be adjusted by flushing the channels 102 with suitable solutions, such as bovine serum albumin (BSA) or a surface-functionalizing solution (step 350). Depending upon the application, proteins typically found in extracellular matrix (ECM), such as collagen, laminin, fibronectin, or elastin, may be attached to the walls via surface functionalization methodologies. The micro-device can then be incorporated into an experimental setup by fitting tubing to the inlets and outlets, connecting the device to other apparatuses, etc. Finally, cells may be seeded and cultured in the channels 102, resulting in an artificial microvascular network

Embodiments of the invention are by no means limited to fabrication by the foregoing exemplary method. Alternate methods for producing the artificial vasculoid microdevices described herein include the use of alternative techniques for making the master molds (e.g., wet etching, plasma etching, or electroplating), and the use of techniques other than replica molding for device construction (e.g., conventional machining, injection molding, or solid freeform fabrication, among other techniques).

### 2. Exemplary Structures Featuring Distensible Walls or Co-Culture Channels

In various embodiments, the invention features devices, and methods of designing, constructing, and implementing the same, that mimic one or more key features of a physiological microvascular structure for the purposes of investigating the interaction between biological or chemical species with each other and with the vessel walls. One such feature includes the ability of the vessel walls to stretch in response to fluid mechanical stimuli, or in response to mechanical transduction effected by cells along the walls of the channels due to chemical or biological stimuli. In vivo, this characteristic, known as vessel distensibility, is an element of the regulatory system for vessel tone. Distensibility may be achieved and engineered with various techniques, which may include using different materials for distensible walls and other, nondistensible components of the microvascular device, varying the specific chemical composition of the employed material, and/or setting the thickness of the walls appropriately.

An exemplary artificial microvascular structure containing a distensible wall is provided by a two-layer-device (as illustrated, for example, in FIGS. 1 and 2), wherein one layer contains the channel pattern, and the second, top layer is sufficiently thin and has a sufficiently low elastic modulus so as to render the channel wall(s) that it forms distensible. For example, for standard PDMS materials, which typically have an elastic modulus of 1.3 MPa or less, distensibility becomes non-negligible for wall thicknesses below about 40 micrometers. In general, the wall thickness is chosen dependent on the elastic modulus of the respective material, and the degree of distensibility desired. In some embodiments, the product of the elastic modulus and the thickness of the walls is less than about 100 Pascal-meters, preferably less than 50 Pascal-meters, more preferably less than 10 Pascal-meters. In various embodiments, the distensible walls comprise an elastomeric material that is different from the material used for the nondistensible parts of the device. Such elastomeric materials include, e.g., polyesteramide, polyglycerol sebacate, polydimethylsiloxane, silk fibroin, and polyurethane, as well as biodegradable materials such as polyoctanediol citrate, polydiol citrate, and polycaprolactone.

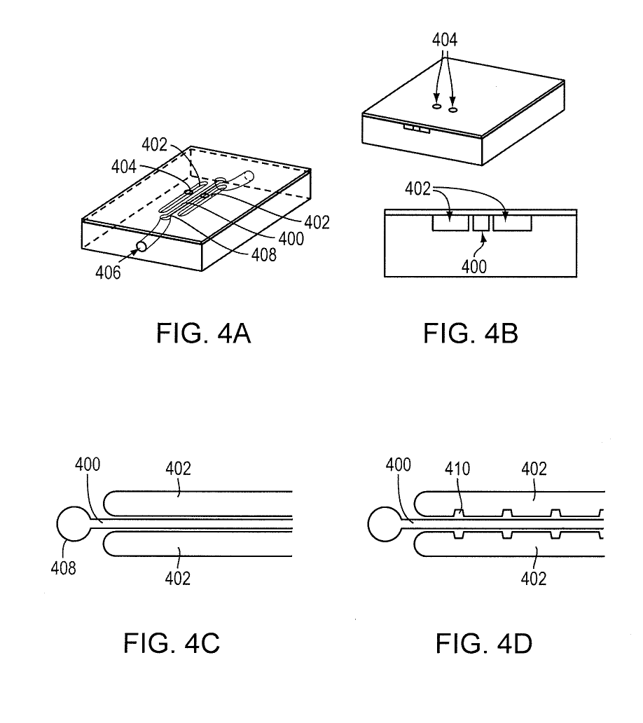

In a modification of the device illustrated in FIGS. 1 and 2, the device may contain three layers, e.g., a middle layer defining the channels, and thin top and bottom sheets providing distensible channel walls. Alternatively or additionally, the side walls between neighboring channels may be distensible, and distensibility may be achieved by a combination of sufficiently small thickness and elastic modulus of the material, analogous to the methods described above for the example of a distensible top wall. More sophisticated structures for artificial microvascular devices, some of which include distensible side walls, are illustrated in FIGS. 4-11 and are described further below. For illustrative purposes, each device features channels without bifurcations, but those of ordinary skill in the art will understand that any of the devices may include networks of channels (e.g., channels with bifurcations). Where two or more channels are employed, they may interact in many ways. For example, a single channel may bifurcate into two channels. In addition, a plurality of channels may be employed in a manifold and be used to connect one or more layers of the device.

FIGS. 4A-D illustrate an exemplary vasculoid device containing a channel 400 with distensible top and side walls. In use, this channel 400 may serve to host one or more cell cultures. Distensibility of the side walls is achieved with additional channels 402 to both sides of channel 400, as indicated in the cross-sectional view of FIG. 4B and the top views of FIGS. 4C and 4D. Channels 402 are vented through pressure relief holes 404 in the thin top layer, and serve as clearance chambers, equilibrating the pressure within the device to the pressure in the environment of the device (typically atmospheric pressure). In this “passive” configuration, distension of the thin walls is caused by internal stresses, for example, as induced by cells seeded within the cell culture channel 400. Typically, the channel 400 is serviced through inlet and outlet channels. FIG. 4A indicates an inlet hole 406 at one end of the inlet channel, and FIGS. 4A, 4C, and 4D illustrate an interface 408 of the inlet channel with the cell culture channel 400 at the other end. In some embodiments, as illustrated in FIG. 4D, the thin walls between channel 400 and clearance chambers 402 have periodically spaced reinforcing ribs 410 that help maintain their upright confirmation despite their small thickness.

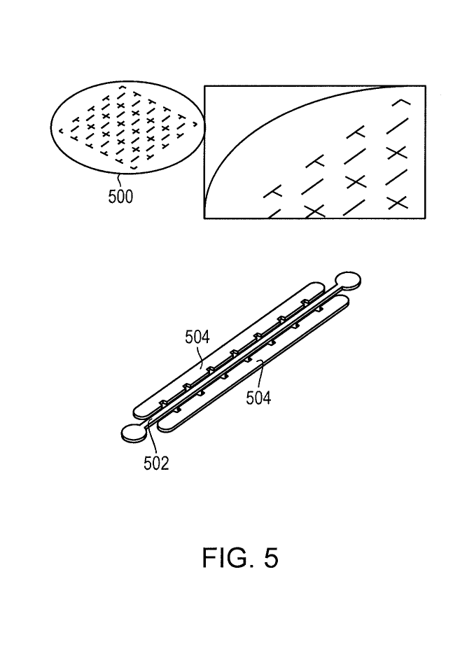

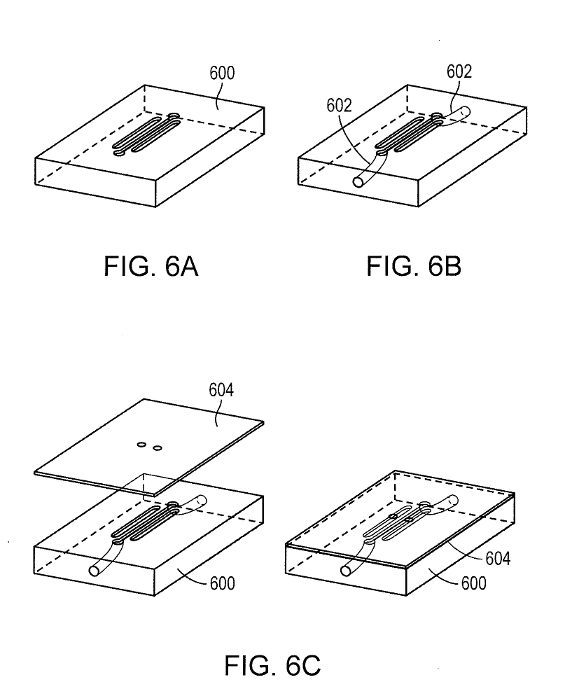

FIG. 5 illustrates a patterned silicon wafer 500, resulting, for example, from sub-process 310 of method 300, which may be used to produce the device depicted in FIG. 4. On the wafer 500, the cell culture channel and clearance chambers appear as relief structures 502 and 504, respectively. In FIG. 6A, the polymer scaffold 600 defining the channels—as produced by replica molding (sub-process 330 of method 300) using wafer 500 as the master mold—is shown. The structure of FIG. 6B further includes inlet and outlet channels 602. These channels may simply be punched mechanically into the device. FIG. 6C illustrates the deposition of a thin polymer sheet 604 with venting holes onto the layer 600. The device may be completed by connecting tubing to the inlet and outlet holes (not shown).

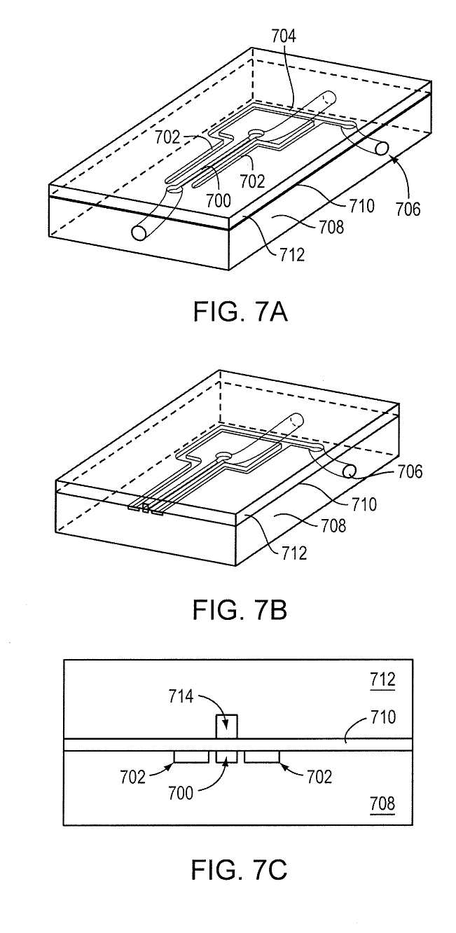

Various embodiments facilitate the application of positive or negative pressure to the walls of the cell culture channel (e.g., channel 700 depicted in FIG. 7) through adjacent channels (e.g., channels 702 depicted in FIG. 7), which, in this case, collectively constitute a pressurizing chamber. This “active” configuration may be of particular use if the walls cannot be made sufficiently compliant to be deformed by cell-induced stresses. FIGS. 7A-7C illustrate an exemplary “active” artificial microvascular device featuring a cell culture channel 700 with three distensible walls. In contrast to the side channels 402 of the device shown in FIG. 4, the pressurizing channels 702 do not contain venting holes, but are instead connected, through a fluidic manifold 704, to each other and to a pressurizing port 706. When the microvascular device is in use, a pressurizing device is connected to the pressurizing port 706. In an exemplary configuration, the pressurizing device may essentially consist of tubing filled, for example, with water, and affixed at an elevated location such as to exert a hydrostatic pressure. Alternatively, more sophisticated devices, such as a positive displacement pump, peristaltic pump, or other device capable of controlling flow rates and pressures in the microfluidic system, can be used to apply a precisely defined positive or negative pressure.

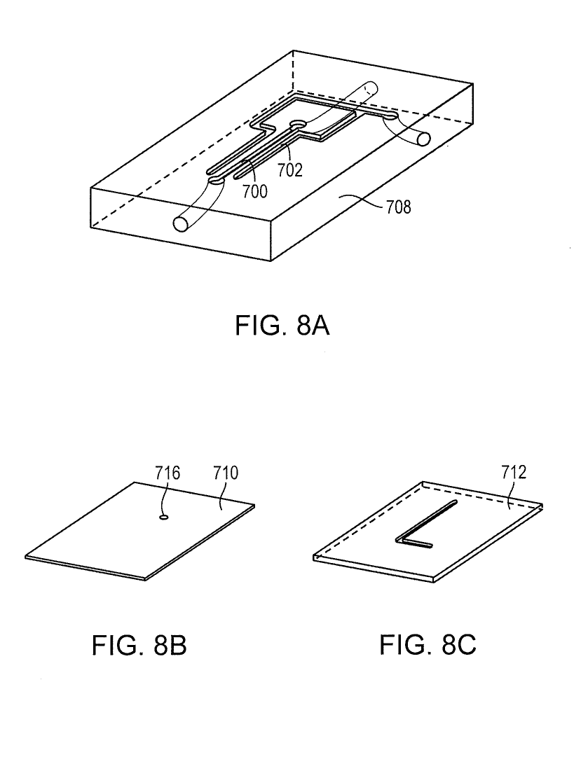

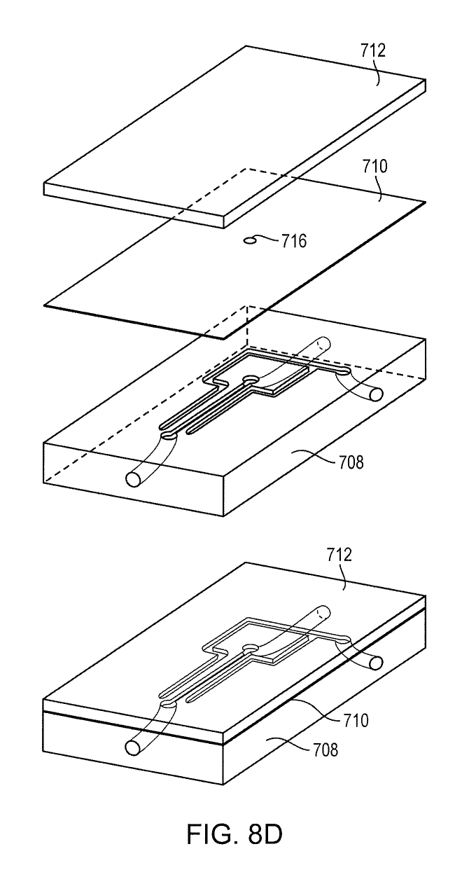

The device shown in FIGS. 7A-7C consists of three layers: a bottom layer 708 defining the cell culture channel 700 and pressurizing channels 702; a thin middle layer 710; and a top layer 712 defining a third pressurizing channel 714 aligned with cell culture channel 702, and separated from channel 702 only through the thin layer 710. The separate layers are depicted in FIGS. 8A-8C. A small hole 716 in the middle layer 710, shown in FIG. 8B, connects the third pressurizing channel 714 to the manifold 704. The relative orientation and assembly of the layers in the microvascular device is illustrated in FIG. 8D.

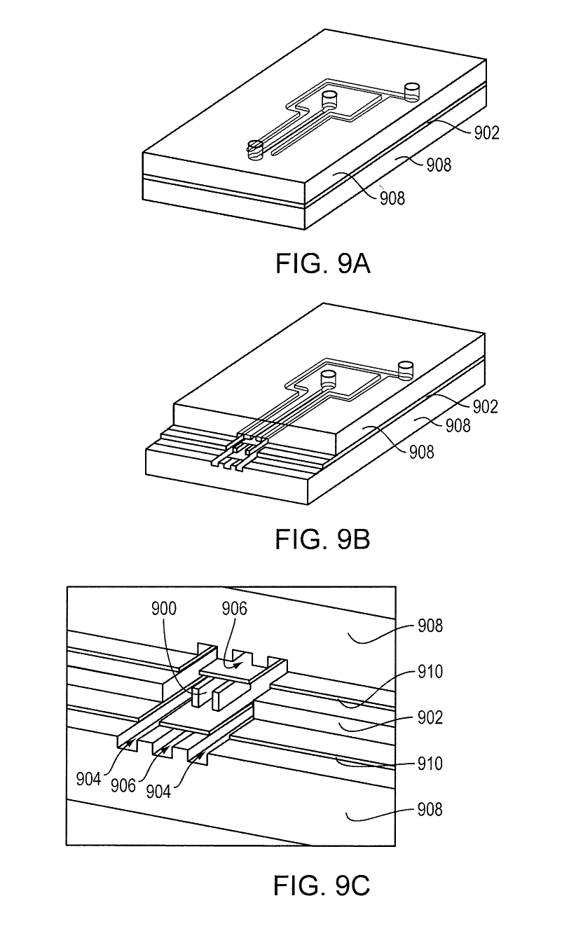

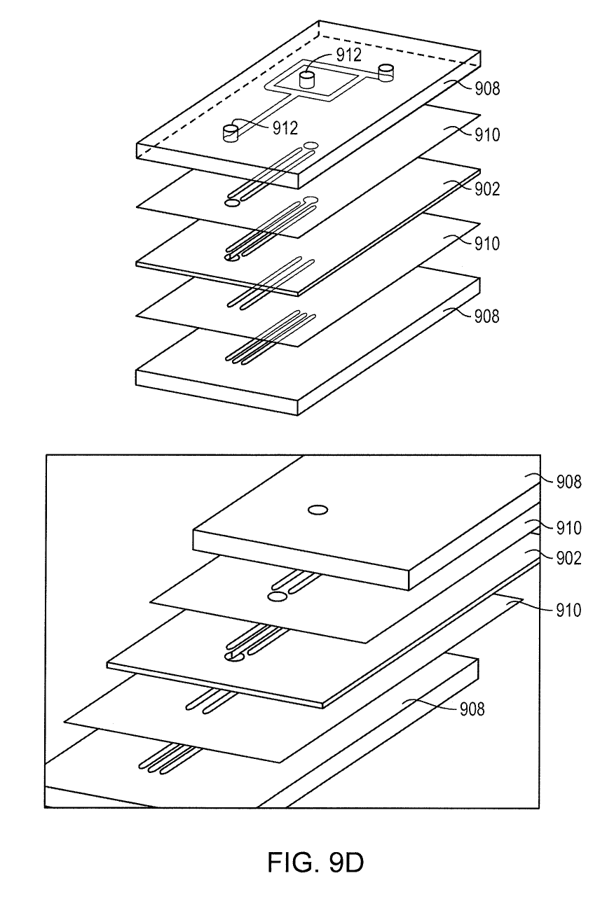

FIGS. 9A-9D illustrate an exemplary device featuring distensible walls in all dimensions. A central cell culture channel 900, located in a middle layer 902 of the device, is surrounded by pressure channels 904 to both sides as well as by top and bottom pressure channels 906 patterned into the top and bottom layers 908. Thin sheets 910 are located between the middle layer 902 and the top and bottom layers 908; i.e., the device comprises five layers. In the illustrated embodiment, inlet and outlet holes 912 of cell culture channel 900 are located in the top layer 908. The device may utilize active pressure channels, or may be implemented in passive configuration by using thinner and/or more flexible walls, and/or by venting the pressure channels to atmospheric pressure.

A second feature that may be provided by the artificial vasculoid described herein is the ability to establish and maintain a robust co-culture condition incorporating the presence of endothelial cells, smooth muscle cells, and other cell types. For endothelial cells, functional or non-functional behavior of the cells may be desired, depending upon the application, and flow properties in the structures may be modulated to control the functional behavior of the cells. For instance, high sustained levels of wall shear stress may be desired to elicit an arterial, functional phenotype, while low, oscillatory shear stresses may be desired to produce an atherogenic phenotype. In biological blood vessels, these cells occupy specific sites within the vessel wall, and are juxtaposed relative to each other as well as to a matrix and blood flow in the intraluminal space. For moderate to larger vessels, structures such as the tunica media, intima, adventitia, and internal elastic lamina are formed; for smaller vessels, the structures are simpler, but a careful interplay between endothelial cells and smooth muscle cells is still observed. Various embodiments of artificial microvascular networks according to the invention implement aspects of such physiological structural organization of cell cultures. For example, the artificial vasculoid may contain two or more channels which are, in large portions, substantially parallel, and separated only by a thin, typically porous or otherwise permeable or semi-permeable wall or membrane. Different cell types may then be seeded into the two or more neighboring channels, and may communicate through the pores in the separating wall. Alternatively or additionally, different cell types may be co-cultured within the same channel(s). The channel walls may, for example, first be lined with one cell type, and subsequently with a second cell type, such as to result in an outer and inner cellular layer. Some methods may involve culturing one cell type, e.g., endothelial cells, inside the lumen of the device, and culturing another cell type, e.g., smooth muscle cells, outside the lumen of the device. Combinations of cell types that may be of particular interest to study include, but are not limited to, endothelial cells and any of smooth muscle cells, pericytes, fibroblasts, and other vascular cells. Dependent upon the tissue of interest, these vascular cells may be further combined with tissue-specific cells, such as, e.g., neurons, adipocytes, dermal cells, epithelial cells, skeletal muscle cells, or bone cells, or with organ-specific cells such as liver cells (e.g., hepatocytes).

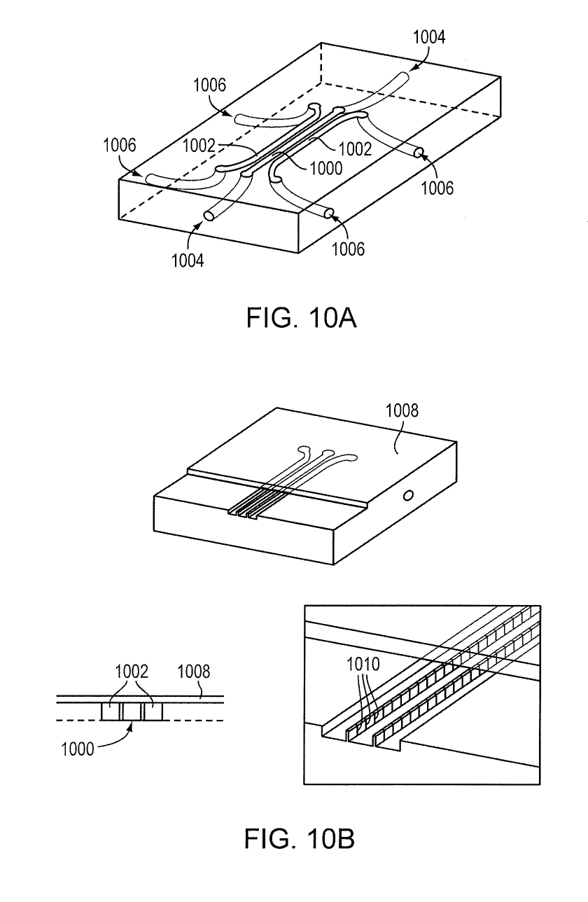

FIGS. 10A-10B illustrate an exemplary approach towards a device containing three cell co-culture channels with fenestrations that allow for mechanical and chemical communication between the channels. As illustrated in FIG. 10A, a center channel 1000 is flanked on either side by co-culture channels 1002, and the center and side channels 1000, 1002 have separate inlets and outlets 1004, 1006, respectively. The two thin walls between the center channel 1000 and the side channels 1002 may consist essentially of wall segments separated by vertical fenestrations 1010, as depicted in FIG. 10B. To preclude cells from wandering between the channels, but allow for the exchange of nutrients, chemical signals, and drug components, the fenestrations may be chosen to be no more than 5 micrometers in width. The embodiment shown constitutes a passive device having a thin top wall 1008 that can stretch due to stresses originating from the cells within the channels 1000, 1002. A co-culture device with top, bottom, and side channels neighboring the central cell-culture channel may also be constructed, for instances, by incorporating aspects of the device illustrated in FIGS. 9A-9D. In this case, permeable walls between the central channel and the top and bottom channels can be achieved by replacing the thin sheet with either off-the-shelf porous membranes or custom fabricated membranes.

Microvascular devices can readily combine the incorporation of several co-culture channels with distensible walls in passive and active configuration. FIGS. 10A-B provide the structure of a passive device with a distensible top wall. It should be noted that the side walls between the co-culture channels may also be distensible if the fenestrated walls are sufficiently thin and flexible. Distension through pressure in the center channel may be achieved if the resistance to fluid flow of the fenestrations is high enough.

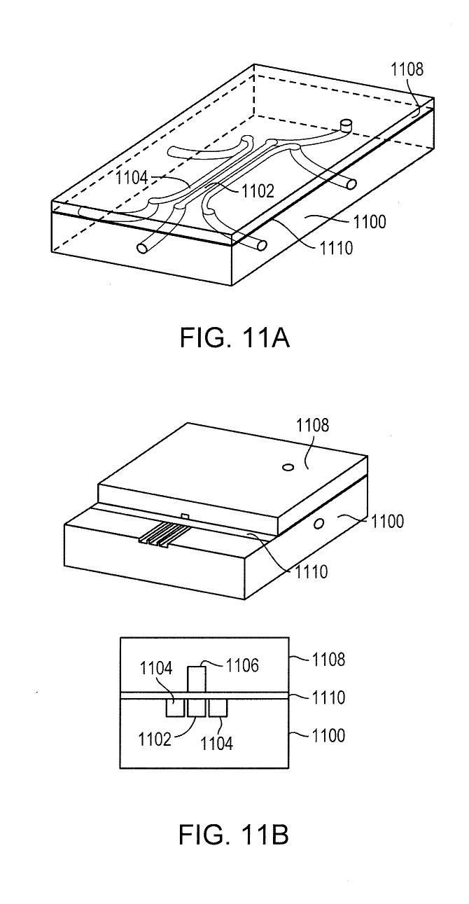

FIGS. 11A-11B illustrate an embodiment of an active device with co-culture channels. This device comprises two layers patterned with channel structures, and a thin sheet connecting them. In a bottom layer 1100, a center channel 1102 is flanked by two co-culture channels 1104. A top layer 1108 contains a pressure channel 1106, connected to a pressurizing port, which is aligned to the center channel 1102, and transfers pressure on a thin wall 1110 in between channels 1102 and 1106.

Possible permutations on the basic structure of the artificial vasculoid are numerous. The following table summarizes structural components that can be employed to render any of the top, side, or bottom walls of a central cell-culture channel distensible and/or (semi-)permeable for co-culture purposes.

| Face of the Center Cell | Construct for a | Construct for Co-culture on |
|---|---|---|
| Culture Channel | Distensible Face | the Other Side of the Face |
| Top | Thin sheet | Off-the-shelf membrane |
| | | or cast porous layer |
| Bottom | Thin sheet | Off-the-shelf membrane |
| | | or cast porous layer |
| Left & Right | Thin walls | Fenestrated walls of |
| | | vertical pores |

### 3. Exemplary Application of Microvascular Devices

In order to use the exemplary devices described above, or similar embodiments, as artificial microvascular structures, the channels are seeded with cells. For this purpose, the inner surface of the polymer scaffolding may be functionalized with molecules that promote cell adhesion to polymer surfaces. These molecules may include, but are not limited to, components of the natural extracellular matrix (ECM), such as collagen, laminin, and fibronectin, or peptide sequences from these molecules, or combinations thereof. Surface functionalization may be achieved by adsorption of the adhesion-promoting molecule(s) to the channel surface, or by covalent chemical linkage of the molecule(s) to the channel. Suitable methods for covalent linkage of biologically active molecules to polymer surfaces are described, for example, in Diaz-Quijada and Wayner, Langmuir 2004 20(22):9607-11, and in Keegan et al., Macromolecules 2004 37(26):9779-84, both of which are hereby incorporated herein by reference in their entirety. In addition, the artificial vasculoid may include a microfluidic structure amenable to cell seeding and to the maintenance of fluid mechanical conditions consistent with physiologic parameters, such as flow velocity and shear stress. Such structures may comprise a steady infusion or pulsatile waveform pump that provides specific levels of flow, pressure, and shear as a function of time to the artificial vasculoid. Depending upon the fluidic resistance and capacitance of the artificial vasculoid and the remainder of the fluid circuit, the input may be modulated in specific ways to control the fluid mechanical interaction between the fluids and the cells.

In various embodiments, artificial microvasculoids as described herein can readily be integrated into conventional imaging modalities. This feature allows for the monitoring of the health or pathology of the vascular structure, as well as its response to mechanical and chemical stimuli in real-time. In some embodiments, the scaffolding used to generate the artificial vasculoid is transparent and relatively thin (e.g., less than 1 mm), and the surface of the vessels is relatively planar with respect to incident illumination. Such devices can be mounted on any light microscope, including transmission, fluorescence, phase-contrast, or confocal microscopes. Movements of the channel walls or cells can then be viewed by eye, or recorded with a CCD camera or similar image recording device. Typically, methods involving fluorescent labeling of cells, or addition of fluorescent beads for tracking purposes, are utilized to provide data on flow rates, streamlines, and other fluid dynamic parameters for the system.

With the exemplary artificial vasculoids described herein, a variety of physiological vascular phenomena can be studied. For example, the artificial vascular structures have the capacity to mimic the results of vascular injury. Vascular injury in vivo often generates breaks in the vessel wall and subsequent leakage of fluid and solid components of the blood into the interstitial space. If an artificial vasculoid were to include smooth, solid walls outside the endothelial cell/smooth muscle cell co-culture, the device would present a non-physiologic barrier against such vessel wall fenestrations. Various embodiments of the present invention, on the other hand, contain openings in the vessel walls, such as small pores or fenestrations, which provide for uninterrupted cell coverage along the inner lumen, while also allowing for vascular tissue breakage and subsequent leakage once the tissue is compromised in response to a vascular injury signal. For experiments conducted using static culture in a petri dish, endothelial cells are not subjected to the fluid mechanical forces they experience in natural blood vessels, and, therefore, conventional cell culture is of limited utility in the investigation of vascular injury. Since the cells are not exhibiting a fully functional, sustainable phenotype in their baseline condition, interactions of these cells with drugs will be of limited utility as well. By contrast, using an artificial vasculoid according to an embodiment of the invention, one may reproduce the physiologic flow conditions seen in natural blood vessels, and, therefore, the interactions with drugs will be more representative of what happens in the body during the process of vascular injury.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An artificial microvascular device, comprising:
a polymer substrate defining:
a first cell culture channel comprising a first inlet and a first outlet, wherein a wall of the first cell culture channel is a cell culturing surface;
a first pressurizing channel comprising a first pressurizing port and separated from the first channel by a first distensible wall;
a second pressurizing channel comprising a second pressurizing port and separated from the first channel by a second distensible wall; and
a pressurizing device coupled to the first pressurizing port and the second pressurizing port and configured to control a pressure level within the first pressurizing channel and the second pressurizing channel to control a deformation of the first distensible wall and the second distensible wall.

2. The device of claim 1, further comprising a second cell culture channel comprising a second cell culturing surface.

3. The device of claim 2, wherein the second cell culture channel is separated from the first cell culture channel by a porous membrane.

4. The device of claim 3, wherein the porous membrane forms a third distensible wall.

5. The device of claim 1, wherein the first pressurizing channel further comprises a first pressurizing outlet and the second pressurizing comprises a second pressurizing outlet.

6. The device of claim 1, wherein the first pressurizing port and the second pressurizing port are coupled together through a fluidic manifold.

7. The device of claim 1, wherein the cell culturing surface is functionalized with molecules that promote cell adhesion to the wall.

8. The device of claim 1, wherein the pressurizing device is one of a displacement pump, a peristaltic pump, and a vacuum pump.

9. The device of claim 1, wherein the pressure level is pulsatile.

10. The device of claim 1, wherein the polymer substrate comprises a material selected from the group consisting of polystyrene, polyesteramide, polyglycerol sebacate, polydimethylsiloxane, polycarbonate, silk fibroin, polyurethane, polyoctanediol citrate, polydiol citrate, and polycaprolactone.

11. The device of claim 1, wherein the first and second distensible walls comprise an elastomeric material selected from the group consisting of polyesteramide, polyglycerol sebacate, polydimethylsiloxane, silk fibroin, polyurethane, polyoctanediol citrate, polydiol citrate, and polycaprolactone.

12. The device of claim 1, further comprising reinforcing ribs attached to the first distensible wall and the second distensible wall.

13. A method comprising:
seeding a plurality of cells onto a wall of a first cell culture channel of an artificial microvascular device, the first cell culture channel comprising a first inlet and a first outlet;
controlling, with a pressurizing device, a first deformation of a first distensible wall of the first cell culture channel by pressurizing, with the pressurizing device, a first pressurizing channel of the artificial microvascular device separated from the first cell culture channel by the first distensible wall;
controlling, with the pressurizing device, a second deformation of a second distensible wall of the first cell culture channel by pressurizing, with the pressurizing device, a second pressurizing channel of the artificial microvascular device separated from the first cell culture channel by the second distensible wall; and
flowing a fluid through the first cell culture channel.

14. The method of claim 13, further comprising seeding cells onto a second cell culturing surface of a second cell culture channel of the artificial microvascular device.

15. The method of claim 14, wherein the second cell culture channel is separated from the first cell culture channel by a porous membrane.

16. The method of claim 13, further comprising generating a pulsatile pressure wave in the first pressurizing channel and the second pressurizing channel.

17. The method of claim 13, wherein the first pressurizing channel further comprises a first pressurizing outlet and the second pressurizing comprises a second pressurizing outlet.

18. The method of claim 13, wherein the first pressurizing port and the second pressurizing port are coupled together through a fluidic manifold.

19. The method of claim 13, further comprising functionalizing the cell culturing surface with molecules that promote cell adhesion to the wall.

20. The method of claim 13, wherein the polymer substrate comprises a material selected from the group consisting of polystyrene, polyesteramide, polyglycerol sebacate, polydimethylsiloxane, polycarbonate, silk fibroin, polyurethane, polyoctanediol citrate, polydiol citrate, and polycaprolactone.

21. The method of claim 13, wherein the first and second distensible walls comprise an elastomeric material selected from the group consisting of polyesteramide, polyglycerol sebacate, polydimethylsiloxane, silk fibroin, polyurethane, polyoctanediol citrate, polydiol citrate, and polycaprolactone.
